(19) United States
(12) Patent Application Publication
(10) Pub. No.: US 2011/0257680 A1
(43) Pub. Date: Oct. 20, 2011

(54) Surgical Forceps Including Pulley Blade Reverser Mechanism

(75) Inventors: Arlen J. Reschke, Longmont, CO (US); Jeffrey M. Roy, Boulder, CO (US); Daniel A. Joseph, Golden, CO (US)

(73) Assignee: TYCO Healthcare Group LP

(21) Appl. No.: 12/763,900

(22) Filed: Apr. 20, 2010

(51) Int. Cl. A61B 17/28

(52) U.S. Cl. 606/206

(57) ABSTRACT

A forceps includes first and second shafts each having a jaw disposed at an end thereof. At least one jaw is moveable from an open to a closed position for grasping tissue therebetween. At least one jaw includes a blade slot defined therein and extending therealong for reciprocation of a blade therethrough. An actuation assembly is disposed within one of the shafts and is configured for translating the blade between a retracted and an extended position. The blade extends at least partially through the blade slot in the extended position. The actuation assembly includes an actuator extending from the shaft. A compliance member couples the actuator to a cable disposed within the shaft. A blade holder engages the cable and has the blade disposed at an end thereof. At least one pulley is coupled to the cable such that translating the actuator proximally translates the blade distally.

### BACKGROUND

The present disclosure relates to a surgical forceps and, more particularly, to a surgical forceps including a pulley-like blade reverser mechanism.

### TECHNICAL FIELD

A forceps is a plier-like instrument which relies on mechanical action between its jaws to grasp, clamp and constrict vessels or tissue. Electrosurgical forceps utilize both mechanical clamping action and electrical energy to affect hemostasis by heating tissue and blood vessels to coagulate and/or cauterize tissue. Certain surgical procedures require more than simply cauterizing tissue and rely on the unique combination of clamping pressure, precise electrosurgical energy control and gap distance (i.e., distance between opposing jaw members when closed about tissue) to “seal” tissue, vessels and certain vascular bundles.

Typically, once a vessel is sealed, the surgeon has to accurately sever the vessel along the newly formed tissue seal. Accordingly, many vessel sealing instruments have been designed which incorporate a knife or blade member which effectively severs the tissue after forming a tissue seal. However, imprecise separation of tissue may result from, for example, misalignment of the blade member with respect to the sealing line. Blade misalignment may also result in blade overload and/or blade fracture, which may pose problems to the user.

### SUMMARY

In accordance with the present disclosure, a forceps is provided. The forceps includes first and second shaft members each having a jaw member disposed at a distal end thereof. One or both of the jaw members is moveable from an open position to a closed position for grasping tissue therebetween. One or both jaw members includes a blade slot defined therein and extending longitudinally therealong that is configured for reciprocation of a blade therethrough. An actuation assembly is disposed within one shaft member and is configured for selectively translating the blade between a retracted position and an extended position. The blade extends partially, or entirely, through the blade slot in the extended position. The actuation assembly includes an actuator extending from the shaft member. A compliance member couples the actuator to a cable disposed within the shaft member. A blade holder also mechanically engages the cable and includes the blade disposed at a distal end thereof. One or more pulleys is operably coupled to the cable such that translating the actuator proximally translates the blade distally to the extended position.

In one embodiment, the forceps includes one or more biasing members for biasing the blade in the retracted position and/or a return spring coupled to the blade holder for returning the blade back to the retracted position.

The compliance member may include a shear pin and/or a compression spring. The shear pin may define a pre-determined load limit such that when a force on the blade exceeds the pre-determined load limit, the shear pin disengages the actuator from the cable. When the actuator is disengaged from the cable, translating the actuator proximally no longer translates the blade distally. The compression spring is compressible in response to a load on the blade such that the compression spring absorbs a portion of the load on the blade and thereby reduces the load on the blade.

In yet another embodiment, the pulley(s) is rotatably mounted within a sleeve disposed within the shaft.

In still another embodiment, the cable defines a loop that is rotatable about first and second pulleys. The first and second pulleys may each define a diameter where the diameter of the first pulley is different from the diameter of the second pulley. Alternatively, the first and second pulleys may define substantially similar diameters.

In still yet another embodiment, the cable includes a nylon coating and/or is made from stainless steel.

In another embodiment, the actuator and/or the blade holder are coupled to the cable in a two-way engagement, such that translating the actuator distally translates the blade proximally back to the retracted position.

In accordance with another embodiment of the present disclosure, an actuation assembly is provided. The actuation assembly is configured for use with a forceps and includes an actuator configured for selectively translating a blade between a retracted position and an extended position. A shear pin defining a pre-determined load limit couples the actuator to a cable loop. A blade holder is coupled to the cable loop and has the blade disposed at a distal end thereof. One or more pulleys is operably coupled to the cable such that translating the actuator proximally translates the blade distally to the extended position. However, when a force on the blade exceeds the pre-determined load limit, the shear pin disengages the actuator from the cable such that translating the actuator proximally no longer translates the blade distally.

In accordance with yet another embodiment of the present disclosure, another actuation assembly is provided. The actuation assembly is configured for use with a forceps and includes an actuator configured for selectively translating a blade between a retracted position and an extended position. A compression spring couples the actuator to a cable loop. A blade holder is coupled to the cable loop and has the blade disposed at a distal end thereof. One or more pulleys is operably coupled to the cable such that translating the actuator proximally translates the blade distally to the extended position. The compression spring is compressible in response to a load on the blade such that, when compressed, the compression spring absorbs at least a portion of the load on the blade and thereby reduces the load on the blade.

### BRIEF DESCRIPTION OF THE DRAWINGS

Various embodiments of the subject instrument are described herein with reference to the drawings wherein:

FIG. 1 is a side, perspective view of a forceps according to an embodiment of the present disclosure;

FIG. 2 is a side, perspective view of the forceps of FIG. 1 with a portion of a handle removed to show the internal components therein;

FIG. 3 is a top view of a jaw member of the forceps of FIG. 1;

FIG. 4 is a schematic illustration of an actuation assembly of the forceps of FIG. 1;

FIG. 5 is a schematic illustration of another embodiment of an actuation assembly of the forceps of FIG. 1 showing a compliance member;

FIG. 6 is a schematic illustration of another embodiment of an actuation assembly of the forceps of FIG. 1 showing a compliance member; and

FIG. 7 is a schematic illustration of another embodiment of an actuation assembly of the forceps of FIG. 1, with parts separated.

### DETAILED DESCRIPTION

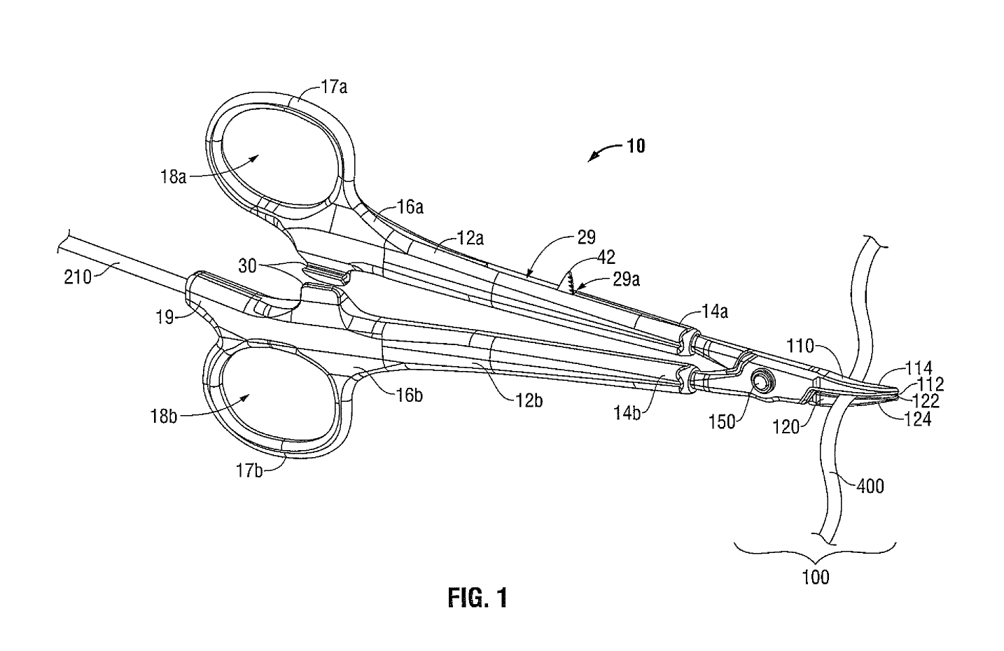

Referring initially to FIG. 1, a forceps 10 includes two elongated shafts 12a and 12b each having a proximal end 16a and 16b and a distal end 14a and 14b, respectively. In the drawings and in the descriptions which follow, the term “proximal,” as is traditional, will refer to the end of the forceps 10 that is closer to the user, while the term “distal” will refer to the end that is further from the user.

The forceps 10 includes an end effector assembly 100 attached to distal ends 14a and 14b of shafts 12a and 12b, respectively. As will be explained in more detail below, the end effector assembly 100 includes a pair of opposing jaw members 110 and 120 that are pivotably connected about a pivot pin 150.

Each shaft 12a and 12b includes a handle 17a and 17b disposed at the proximal end 16a and 16b thereof. Each handle 17a and 17b defines a finger hole 18a and 18b therethrough for receiving a finger of the user. As can be appreciated, finger holes 18a and 18b facilitate movement of the shafts 12a and 12b relative to one another that, in turn, pivots the jaw members 110 and 120 from an open position, wherein the jaw members 110 and 120 are disposed in spaced-apart relation relative to one another, to a closed position (FIG. 1), wherein the jaw members 110 and 120 cooperate to grasp tissue 400 therebetween.

A ratchet 30 may be included for selectively locking the jaw members 110 and 120 relative to one another at various positions during pivoting. It is envisioned that the ratchet 30 may include graduations or other visual markings that enable the user to easily and quickly ascertain and control the amount of closure force desired between the jaw members 110 and 120.

With continued reference to FIG. 1, one of the shafts, e.g., shaft 12b, includes a proximal shaft connector 19 which is designed to connect the forceps 10 to a source of electrosurgical energy such as an electrosurgical generator (not shown). Proximal shaft connector 19 secures an electrosurgical cable 210 to the forceps 10 such that the user may selectively apply electrosurgical energy as needed.

As mentioned above, the two opposing jaw members 110 and 120 of the end effector assembly 100 are pivotable about pivot pin 150 from the open position to the closed position for grasping tissue 400 therebetween. Jaw member 110 includes an insulated outer housing 114 that is dimensioned to mechanically engage an electrically conductive sealing surface 112 of jaw member 110. Similarly, jaw member 120 includes an insulated outer housing 124 that is dimensioned to mechanically engage an electrically conductive sealing surface 122 of jaw member 120. Electrically conductive sealing surfaces 112 and 122 are opposed to one another such that, upon activation, electrosurgical energy may be supplied to the electrically conductive sealing surfaces 112 and 122 to seal tissue disposed between the jaw members 110 and 120.

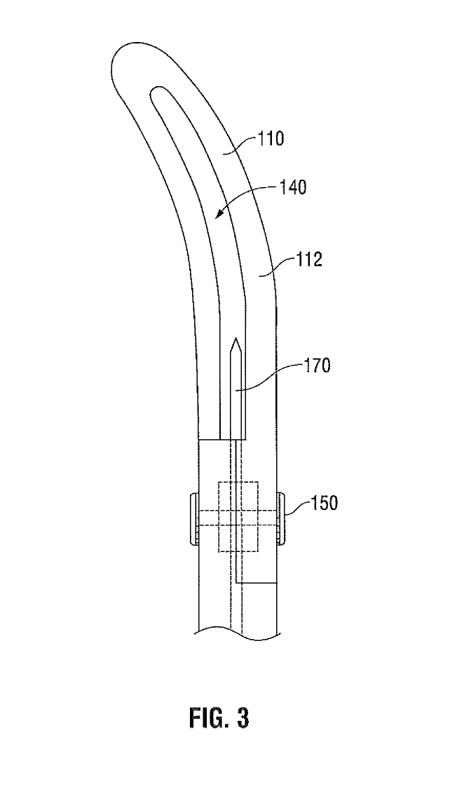

As best seen in FIG. 3, jaw member 110 includes a blade slot, or blade channel 140 extending therethrough. The blade channel 140 is configured for reciprocation of a cutting mechanism, e.g., a blade 170, therethrough. As shown, blade channel 140 is defined completely within jaw member 110. However, the blade channel 140 may be formed when two opposing blade channels defined within jaw members 110 and 120 come together upon pivoting of the jaw members 110 and 120 to the closed position. Further, the blade channel 140 may be configured to facilitate and/or enhance cutting of tissue during reciprocation of the cutting blade 170 in the distal direction.

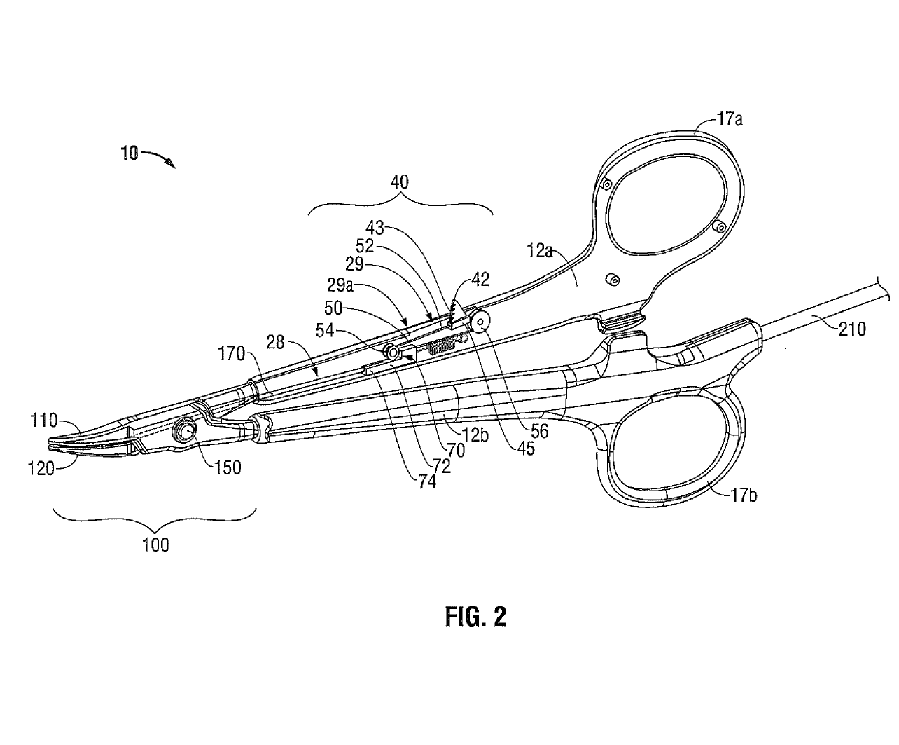

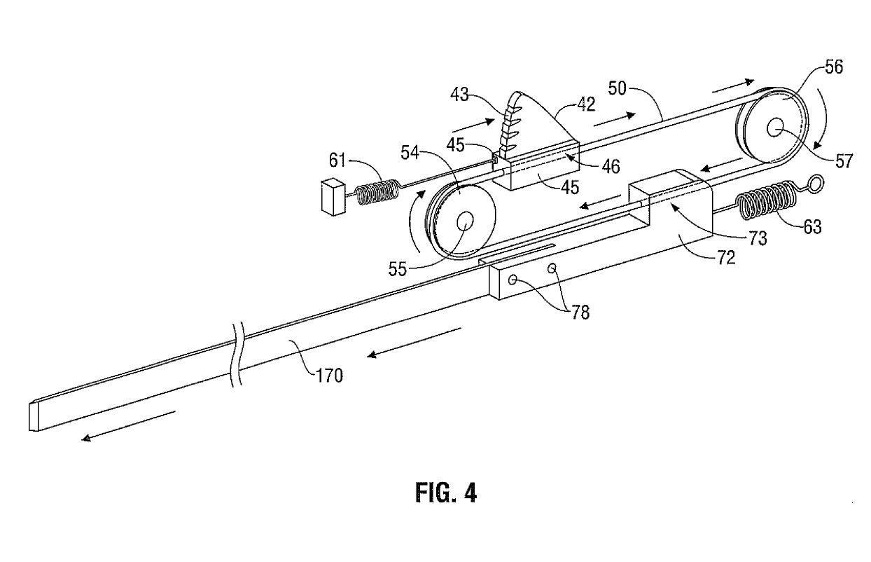

Referring now to FIG. 2, the arrangement of shaft 12a is slightly different from shaft 12b. As shown in FIG. 2, shaft 12a is hollow to define a chamber 28 therethrough that is configured to house an actuation assembly 40 and a blade assembly 70 therein. Blade assembly 70 includes a blade holder 72 having blade 170 disposed at a distal end 74 thereof. Blade 170 may be integral with blade holder 72, or may be attached thereto by other suitable mechanisms, e.g., by a plurality of pins 78 (FIG. 4) disposed through both blade 170 and blade holder 72. As will be described in detail below, blade 170 is translatable through shaft 12a and at least partially into blade channel 140 (FIG. 3) to cut tissue 400 disposed between jaw members 110 and 120.

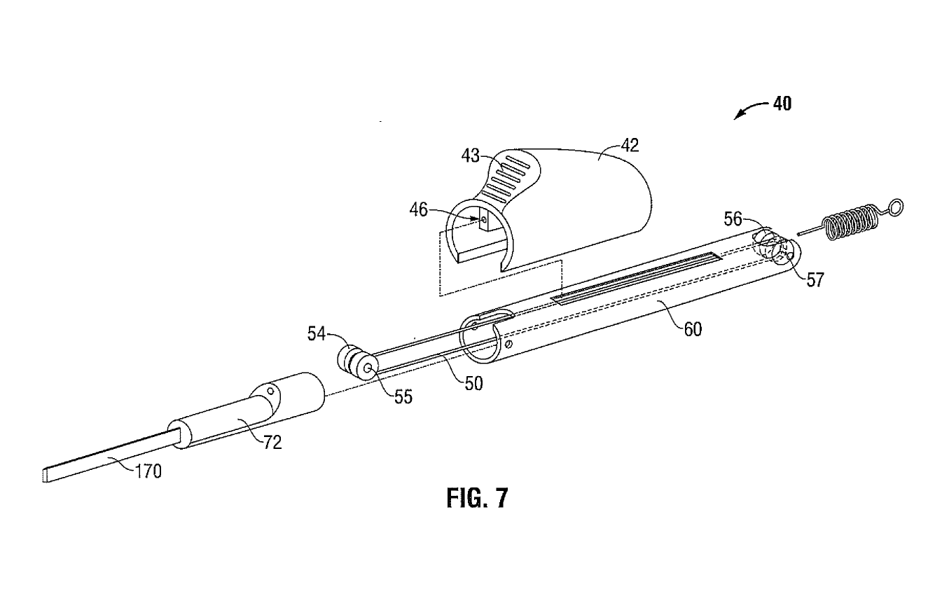

With reference now to FIGS. 2 and 4, actuation assembly 40 includes an actuator 42 having a finger tab and a base 45 that defines a lumen 46 therethrough. The actuator 42 is slidable with respect to shaft 12a. A slot 29 is defined within shaft 12a to permit longitudinal translation of actuator 42 with respect to shaft 12a. A cable 50 is disposed through lumen 46 of actuator 42 and is secured therein to engage actuator 42 to cable 50. Cable 50 defines a loop and is disposed about first and second pulleys 54 and 56, respectively. Cable 50 may be formed from stainless steel and/or may include a nylon coating to facilitate rotation about pulleys 54 and 56. Further, as shown in FIG. 7, actuation assembly 40 may be disposed within a sleeve 60 positioned within shaft 12a, with pulleys 54 and 56 being rotatably secured within sleeve 60 via pins 55 and 57, respectively. Although two pulleys 54 and 56 are shown in FIGS. 4 and 7, greater or fewer than two pulleys may also be provided.

Referring again to FIG. 4, a blade holder 72 is disposed on cable 50 opposite actuator 42 with pulleys 54 and 56 therebetween. Cable 50 is disposed through a lumen 73 defined through blade holder 72 to engage blade holder 72 thereon. As can be appreciated, due to the configuration of cable 50, actuator 42, and blade holder 72, proximal translation of actuator 42 causes clockwise rotation of cable 50 about pulleys 54 and 56 and, thus, distal translation of blade holder 72. Further, actuator 42 and blade holder 72 may be coupled to cable 50 in a two-way engagement, such that distal translation of actuator 42 causes counter-clockwise translation of cable 50 about pulleys 54 and 56 and proximal translation of blade holder 72. Accordingly, actuator 42 may be translated proximally to move blade 170 between a retracted position and an extended position such that blade 170 extends into blade channel 140 in the extended position to cut tissue 400 disposed between the jaw members 110 and 120.

As shown in FIG. 4, pulley 54 and pulley 56 have a substantially similar diameter such that translation of actuator 42 proximally translates blade holder 72 distally in a substantially parallel direction. However, pulley 54 and pulley 56 may have different diameters, e.g., as shown in FIG. 2, where pulley 56 has a diameter that is larger than a diameter of pulley 54, such that actuator 42 is translated proximally at a pre-determined angle with respect to the distal translation of blade holder 72.

Referring now to FIGS. 1, 2 and 4, actuator 42 is initially disposed in a distal position (FIG. 1), at a distal end 29a of slot 29 of shaft. At the same time, blade holder 72 is disposed in a proximal position such that blade 170 is disposed completely within shaft 12a, and thus does not extend into blade channel 140. A biasing mechanism 61, e.g. a spring 61, coupled to actuator 42, may be used to bias actuator 42 in the distal position such that blade holder 72 is biased in the proximal, or retracted position. Further, a return spring 63, or other biasing mechanism 63, may be coupled to blade holder 72 to similarly bias blade holder 72 in the retracted position and thus, bias actuator 42 in the distal position. Return spring 63 also acts to return blade holder 72, and thus blade 170, to the retracted position once blade 170 has been deployed. As can be appreciated, a user must overcome the biasing force of biasing spring 61 and/or return spring 63 in order to translate actuator 42 proximally and thereby advance blade 170 distally through blade channel 140. Similarly, when the proximal force applied to finger tab 43 is removed, e.g., when the actuator 42 is released, blade 170 is returned to the retracted position under the bias of return spring 63. Such a configuration acts as a safety feature that prevents blade 170 from being inadvertently left in the extended position. Additionally, as mentioned above, actuator 42 may be translated distally to manually return blade holder 72 and blade 170 to the retracted position. Manual return of blade 170 may be necessary, for example, if blade 170 becomes lodged or jammed in the extended position.

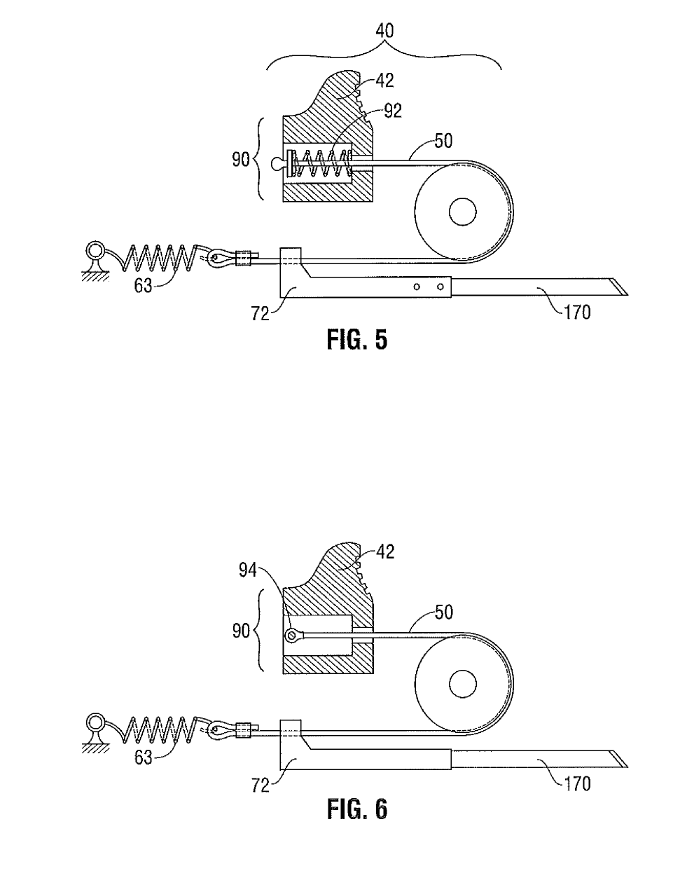

With reference now to FIGS. 5 and 6, when a user translates actuator 42 proximally to the position shown in FIG. 2, blade 170 is advanced through tissue disposed between jaw members 110 and 120. However, during advancement of blade 170 through tissue, specific portions of tissue may impede passage of blade 170 more than others. In other words, the force required to urge blade 170 through blade channel 140 may vary depending on the composition and/or size of tissue to be cut. As the user translates the actuator 42 proximally, blade 170 is urged into tissue and tissue resists translation of blade 170 therethrough. This resistance imparts a load on the blade 170. If the load is great enough, the blade 170 may become misaligned due to overload and/or may fracture. Accordingly, a compliance feature 90 may be included within the actuation assembly 40 to limit blade overload and/or prevent blade fracture. More specifically, the actuator 42 may be engaged to the cable 50 via a compression spring 92 and/or a shear pin 94. As tissue resists distal translation of blade 170, and as cable 50 urges blade holder 72 distally, a load is imparted to blade 170. A user, unaware of the load on blade 170, may attempt to translate actuator 42 further proximally to advance blade 170 further through tissue, which increases the load on blade 170. The compression spring 92 would compress in response to the added load and absorb some of the load on blade 170, thereby decreasing the possibility of blade overload.

With respect to FIG. 6, a shear pin 94, either in conjunction with, or in place of, the compression spring 92, may be provided to define a pre-determined load limit. This pre-determined load limit would cause the shear pin 94 to shear, thereby disengaging actuator 42 from cable 50 when overloading occurs. Accordingly, instead of blade 170 being urged into tissue to the point of fracture, actuator 42 would automatically disengage from cable 50 such that translation of actuator 42 no longer affects translation of the blade through tissue, thereby removing the load from blade 170 and helping to prevent blade fracture. The pre-determined load limit would necessarily correspond to a load that is below the blade fracture point. In other words, the pre-determined load limit would disengage actuator 42 from cable 50 prior to blade fracture. Further, return spring 63, in conjunction with compression spring 92 and/or shear pin 94 would help ensure that, in the event of blade disengagement from cable 50, blade 170 and any corresponding debris would remain inside shaft 12a of forceps 10, and would not compromise the surgical site, which may not be the case if the blade 170 fractures.

A compliance member (not shown), e.g., a compression spring and/or a shear pin, may be provided to couple blade holder 72 to cable 50. In this configuration, blade holder 72 would disengage from cable 50 in response to a load exceeding the pre-determined load limit of the shear pin. As with the previous embodiment, the compression spring 92 would act to absorb some of the load, thereby reducing the load on blade 170.

Forceps 10 may also include a lockout mechanism (not shown) for preventing accidental reciprocation of blade 170 through blade channels 140a and 140b. Such a feature would prevent blade 170 from being translated distally until the jaw members 110 and 120 are disposed in the closed position. The lockout mechanism may include mechanical components and/or electrical components, such as a sensor.

With reference now to FIGS. 1-7, the operation of forceps 10 is described. Initially, forceps 10 is positioned such that jaw members 110 and 120 are spaced-apart relative to one another with tissue 400 disposed therebetween. At this point, the lockout mechanism may be used to prevent inadvertent deployment of blade 170 until jaw members 110 and 120 are moved to the closed position. Once positioned as desired, a user may engage finger holes 18a and 18b to squeeze shafts 12a and 12b together, such that jaw members 110 and 120 are moved from the spaced-apart to the closed position, grasping tissue 400 therebetween. As discussed above, ratchet 30 may selectively lock the jaw members 110 and 120 relative to one another at various positions during pivoting, such that the desired force may be applied accurately and consistently to tissue 400. The user may then selectively apply electrosurgical energy to electrically conductive sealing plates 112 and 122 of jaw members 110 and 120, respectively, to thereby effectuate a tissue seal.

Once tissue has been adequately sealed, the user may translate finger tab 43 of actuator 42 proximally, thereby rotating cable 50 about pulleys 54 and 56 and advancing blade 170 distally from shaft 12a through blade channel 140 defined within jaw members 110 and 120 to cut tissue 400 therebetween. When blade 170 has been advanced sufficiently through blade channel 140 to cut tissue disposed between jaw member 110 and 120, finger tab 43 may be released by the user. Actuator 42 will then return to the initial, distal position, while blade 170 returns to the initial, proximal position under the bias of biasing spring 61 and/or return spring 63. If the blade 170 is prevented from returning under a bias, e.g., due to tissue and/or debris blockage, the user may manually translate finger tab 43 in the distal direction to retract blade holder 72 and blade 170 back to the retracted position. Once tissue 400 has been sealed and cut, the user may move the finger holes 18a and 18b apart from one another to open jaw members 110 and 120 such that the forceps 10 may be removed from the surgical site.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps comprising:
first and second shaft members each having a jaw member disposed at a distal end thereof, at least one of the jaw members moveable from an open position to a closed position for grasping tissue therebetween, at least one of the jaw members including a blade slot defined therein and extending longitudinally therealong, the blade slot configured for reciprocation of a blade therethrough; and
an actuation assembly disposed within one of the shaft members, the actuation assembly configured for selectively translating the blade between a retracted position and an extended position wherein the blade extends at least partially through the blade slot in the extended position, the actuation assembly including:
an actuator;
a compliance member coupling the actuator to a cable disposed within the shaft member;
a blade holder mechanically engaging the cable and having the blade disposed at a distal end thereof; and
at least one pulley operably coupled to the cable such that translating the actuator proximally translates the blade distally to the extended position.

2. The forceps according to claim 1, further comprising at least one biasing member for biasing the blade in the retracted position.

3. The forceps according to claim 1, further comprising a return spring coupled to the blade holder for returning the blade back to the retracted position.

4. The forceps according to claim 1, wherein the compliance member includes at least one of a shear pin and a compression spring.

5. The forceps according to claim 4, wherein the shear pin defines a pre-determined load limit wherein when a force on the blade exceeds the pre-determined load limit, the shear pin disengages the actuator from the cable such that translating the actuator proximally no longer translates the blade distally.

6. The forceps according to claim 4, wherein the compression spring is compressed in response to a load on the blade such that the compression spring absorbs at least a portion of the load on the blade and thereby reduces the load on the blade.

7. The forceps according to claim 1, wherein the at least one pulley is rotatably mounted within a sleeve disposed within the shaft.

8. The forceps according to claim 1, wherein the cable defines a loop, the loop being rotatable about first and second pulleys.

9. The forceps according to claim 8, wherein the first and second pulleys each define a diameter, the diameter of the first pulley being different from the diameter of the second pulley.

10. The forceps according to claim 1, wherein the cable includes a nylon coating.

11. The forceps according to claim 1, wherein the cable is made from stainless steel.

12. The forceps according to claim 1, wherein the actuator and the blade holder are coupled to the cable in a two-way engagement such that translating the actuator distally translates the blade proximally back to the retracted position.

13. An actuation assembly configured for use with a forceps, the actuation assembly comprising:
an actuator configured for selectively translating a blade between a retracted position and an extended position;
a shear pin defining a pre-determined load limit coupling the actuator to a cable loop;
a blade holder coupled to the cable loop and having the blade disposed at a distal end thereof;
at least one pulley operably coupled to the cable such that translating the actuator proximally translates the blade distally to the extended position; and
wherein, when a force on the blade exceeds the pre-determined load limit, the shear pin disengages the actuator from the cable such that translating the actuator proximally no longer translates the blade distally.

14. An actuation assembly configured for use with a forceps, the actuation assembly comprising:
an actuator configured for selectively translating a blade between a retracted position and an extended position;
a compression spring coupling the actuator to a cable loop;
a blade holder coupled to the cable loop and having the blade disposed at a distal end thereof;
at least one pulley operably coupled to the cable such that translating the actuator proximally translates the blade distally to the extended position; and
wherein, the compression spring is compressible in response to a load on the blade such that, when compressed, the compression spring absorbs at least a portion of the load on the blade and thereby reduces the load on the blade.
